(19) United States
(12) Patent Application Publication
(10) Pub. No.: US 2020/0352504 A1
(43) Pub. Date: Nov. 12, 2020

(54) Image Drunken Driving Judgment System and Related Method

(71) Applicant: FaceHeart Inc., Hsinchu City (TW)

(72) Inventors: Tsuey-Huey Shiue, Hsinchu City (TW); Yin-Cheng Tsai, Taoyuan City (TW); Kuan-Hung Chen, Hsinchu City (TW); Meng-Liang Chung, Changhua County (TW)

(21) Appl. No.: 16/514,972

(22) Filed: Jul. 17, 2019

(30) Foreign Application Priority Data
May 9, 2019 (TW) 108116017

(51) Int. Cl. A61B 5/00; A61B 5/0205; A61B 5/1455; A61B 5/18; G06N 3/04; G06N 3/08

(52) U.S. Cl. CPC A61B 5/4845; A61B 5/0205; A61B 5/14552; A61B 5/18; A61B 5/02405; A61B 5/7267; A61B 5/7275; G06N 3/0436; G06N 3/08; A61B 5/7278

(57) ABSTRACT

A drunken driving judgment system includes an image capturing module configured to obtain multiple images associated with a subject; a physiological parameter computing module coupled to the image capturing module, and configured to generate at least one physiological parameter according to the multiple images associated with the subject, wherein the at least one physiological parameter comprises at least one of a Remote PhotoPlethysmoGraphy, a heart rate, a heart rate variability, a blood oxygen, a breath rate, and a blood pressure; and an alcohol detection calculation unit coupled to the physiological parameter computing module, and configured to generate a drunken driving judgment result according to the at least one physiological parameter to indicate whether the subject is drunken.

### BACKGROUND OF THE INVENTION

### 1. Field of the Invention

The present invention relates to a drunken driving judgment system and related method, and more particularly, to an image drunken driving judgment system and related method capable of judging a drunken driver according to driver's images.

### 2. Description of the Prior Art

Drunken driving easily leads to accident to others and the driver. How to prevent and catch drunken driving has become an urgent problem to be solved. It is conventional to use a breath alcohol tester to estimate blood alcohol level from a breath sample of a subject according to alcohol level of the breath sample. However, the breath alcohol tester can be done once at one time and cannot continuously monitor the blood alcohol level of the driver. In addition, a precision of the breath alcohol tester is influenced by the volume of the breath sample to be unstable.

Therefore, how to provide a new drunken driving judgment system to improve disadvantages of the breath alcohol tester and quickly and easily perform alcohol test for the driver has become a new topic in the industry.

### SUMMARY OF THE INVENTION

It is therefore an objective of the present invention to provide an image drunken driving judgment system and related method to quickly judge a drunken driver without touching the driver.

The present invention discloses a drunken driving judgment system including an image capturing module configured to obtain multiple images associated with a subject; a physiological parameter computing module coupled to the image capturing module, and configured to generate at least one physiological parameter according to the multiple images associated with the subject, wherein the at least one physiological parameter comprises at least one of a Remote PhotoPlethysmoGraphy, a heart rate, a heart rate variability, a blood oxygen, a breath rate, and a blood pressure; and an alcohol detection calculation unit coupled to the physiological parameter computing module, and configured to generate a drunken driving judgment result according to the at least one physiological parameter to indicate whether the subject is drunken.

The present invention further discloses a drunken driving judgment method including obtaining multiple images associated with a subject; inputting the multiple images associated with the subject to a physiological parameter computing module to generate at least one physiological parameter, wherein the at least one physiological parameter comprises at least one of a Remote PhotoPlethysmoGraphy, a heart rate, a heart rate variability, a blood oxygen, a breath rate, and a blood pressure; and inputting the at least one physiological parameter to an alcohol detection calculation unit to generate a drunken driving judgment result to indicate whether the subject is drunken.

The present invention converts images of the subject into a Remote PhotoPlethysmoGraphy to analyze the physiological parameter including the heart rate, the heart rate variability, the blood oxygen, the breath rate, the blood pressure, and the like, so as to judge whether the subject is drunken. As a result, under the architecture of the drunken driving judgment system, the present invention is able to quickly and easily examine whether the driver is drunken without touching the driver.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a functional block diagram of a drunken driving judgment system according to an embodiment of the present invention.

FIG. 2 illustrates an electrocardiogram and a remote PhotoPlethysmoGraphy according to an embodiment of the present invention.

FIG. 3 illustrates a heart rate variability spectrogram according to an embodiment of the present invention.

FIG. 4 is a flowchart of a drunken driving judgment process according to an embodiment of the present invention.

### DETAILED DESCRIPTION

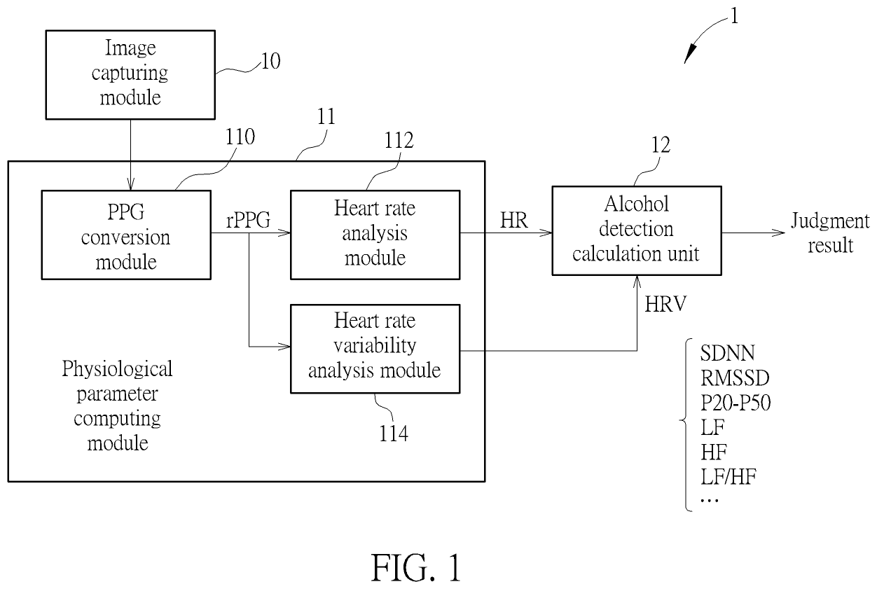

FIG. 1 is a functional block diagram of a drunken driving judgment system 1 according to an embodiment of the present invention. The drunken driving judgment system 1 includes an image capturing module 10, a physiological parameter computing module 11, and an alcohol detection calculation unit 12.

The image capturing module 10 is configured to continuously capturing images of a subject (e.g., continuously capturing images from 3 to 5 minutes), to continuously obtain multiple images associated with the subject and color light information of the multiple images. The image capturing module 13 may be a front lens of an electronic device such as an Internet camera, a built-in camera of a laptop computer or a smart phone, which is not limited.

The physiological parameter computing module 11 is coupled to the image capturing module 10 and the alcohol detection calculation unit 12, and configured to generate at least one physiological parameter to the alcohol detection calculation unit 12 according to the multiple images associated with the subject. The physiological parameter mainly but not limited to include a Remote PhotoPlethysmoGraphy (hereinafter abbreviated rPPG), a heart rate (HR), a heart rate variability (HRV), a blood oxygen, a breath rate, a blood pressure, and the like.

The alcohol detection calculation unit 12 is coupled to the physiological parameter computing module 11, and configured to generate a drunken driving judgment result according to the at least one physiological parameter to indicate whether the subject is drunken. A method of judging drunken driving includes but not limited to fuzzy theory and artificial neural network algorithm. For example, the alcohol detection calculation unit 12 may establish a drunken driving prediction principle using the fuzzy theory in advance according to given characteristics of the physiological parameter, and then the drunken driving judgment result may be obtained by inputting the at least one physiological parameter to the established drunken driving prediction principle; or, the alcohol detection calculation unit 12 may train and establish a drunken driving prediction model using artificial neural network algorithm in advance according to multiple kinds of learning samples, and then the drunken driving judgment result may be obtained by inputting the at least one physiological parameter to the established drunken driving prediction model.

The physiological parameter computing module 11 includes a PPG conversion module 110, a heart rate analysis module 112 and a heart rate variability analysis module 114. The PPG conversion module 110 is coupled to the image capturing module 10, and configured to convert the multiple images associated with the subject into an rPPG. Based on a fact that light sensing elements absorb light energy, the rPPG is a signal that reflects light energy variation due to the rhythmic flow of arterial blood resulting in different levels of light energy absorption, such light energy variation can be detect at the skin surface of the subject. Therefore, it is a non-invasive measuring method having advantages such as with little equipment, easy implementation, and low cost.

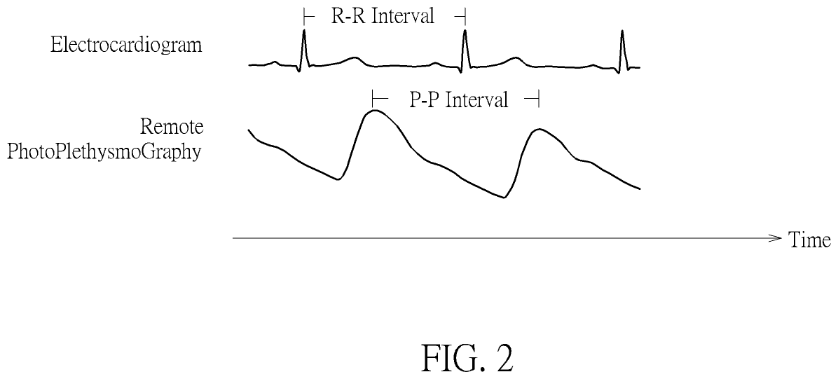

The heart rate analysis module 112 is coupled to the PPG conversion module 110, and configured to judge the heart rate of the subject according to the rPPG. FIG. 2 is a schematic diagram of an electrocardiogram and an rPPG according to an embodiment of the present invention. An interval between one peak to another peak of the electrocardiogram is named as an R-R interval or an interbeat interval (IBI), and the heart rate per minute may be obtained by computing an averaged R-R interval per minute. The averaged heart rate is abbreviated the heart rate, which may be one of the physiological parameter judging whether the subject is drunken. For example, the heart rate of a drunken driver is influenced by the alcohol, so that the heart rate of the drunken driver should be out of a non-drunken range of a non-drunken driver. An interval between one peak to another peak of the rPPG is named as a P-P interval, and the averaged heart rate per minute may be obtained by computing an averaged P-P interval per minute. Therefore, the present invention analyzes the physiological parameter of the driver (i.e., the subject) by measuring the rPPG to replace measuring the conventional electrocardiogram, so as to judge how drunk the subject is without touching the subject.

The heart rate variability analysis module 114 is coupled to the PPG conversion module 110, and configured to judge the heart rate variability of the subject according to the rPPG. In one embodiment, the physiological parameter computing module 11 further includes an analysis module configured to judge the blood oxygen, the breath rate and the blood pressure of the subject according to the rPPG, which is not limited.

In one embodiment, factors that influence the heart rate variability may be classified into a time domain indication and a frequency domain indication. For example, the time domain indication influencing the heart rate variability includes but not limited to a standard deviation of all normal to normal intervals (SDNN), a root mean square successive differences (RMSSD) and a ratio of P20 to P50, wherein P20/P50 refers to a number of adjacent NN (normal to normal) intervals differing by more than 20/50 milliseconds in the entire recording.

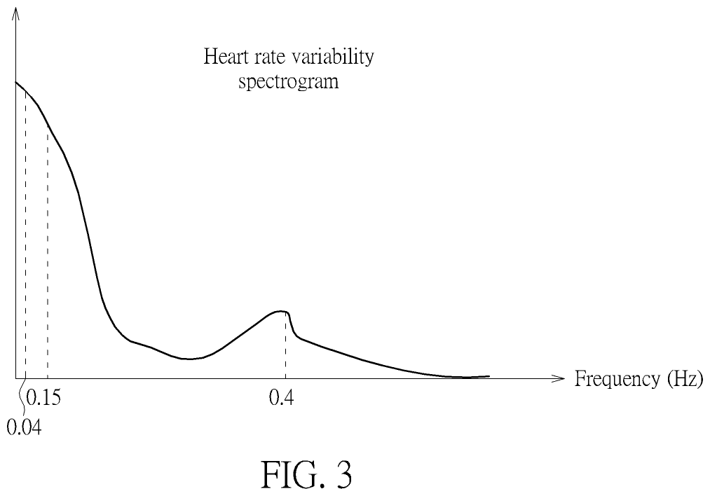

The frequency domain indication influencing the heart rate variability includes but not limited to a low frequency (LF) indication, a high frequency (HF) indication and a ratio of the low frequency and the high frequency (LF/HF). FIG. 3 illustrates a heart rate variability spectrogram according to an embodiment of the present invention. As shown in FIG. 3, the low frequency indication is obtained according to a waveform segment at the frequency range from 0.04 to 0.15 Hz when the rPPG is converted into the frequency domain. The high frequency indication is obtained according to a waveform segment at the frequency range from 0.15 to 0.4 Hz when the rPPG is converted into the frequency domain. The LF/HF ratio is used to indicate a balance of sympathetic or parasympathy nervous system or a control of sympathy nervous system. Specific computations of the time and frequency domain indications may be well known in the art, which is omitted.

In brief, factors that influence the heart rate variability include time domain indications (such as SDNN, RMSSD, P20 to P50) and frequency domain indications (such as LF, HF, LF/HF) , the alcohol detection calculation unit 12 may judge whether the subject is drunken according to the heart rate and indications associated with the heart rate variability, wherein the heart rate and the indications associated with the heart rate variability may be obtained according to the images of the subject. Therefore, under the architecture of the drunken driving judgment system 1, the present invention is able to quickly and easily examine whether the driver is drunken without touching the driver.

For example, the alcohol detection calculation unit 12 may judge how the driver is drunk according to given characteristics of the physiological parameter, e.g., the decrease of the low frequency indication (LF) is highly associated with an amount of alcohol intake, which is not limited. The alcohol detection calculation unit 12 may establish a drunken driving prediction principle using the fuzzy theory and the at least one physiological parameter, and then the drunken driving judgment result may be obtained by inputting the at least one physiological parameter to the established drunken driving prediction principle.

For example, the alcohol detection calculation unit 12 may judge how the driver is drunk according to given learning samples including drunken and sober cases to train and establish a drunken driving prediction model using artificial neural network algorithm in advance, wherein the learning sample includes the physiological parameter that is highly associated with alcohol such as but not limited to the heart rate, the heart rate variability, the blood oxygen, the breath rate and the blood pressure. The alcohol detection calculation unit 12 may obtain the drunken driving judgment result by inputting the at least one physiological parameter to the established drunken driving prediction model.

The abovementioned embodiments regarding the fuzzy theory and the artificial neural network algorithm are examples of the present invention, and practical implementations of the alcohol detection calculation unit 12 are not limited to the abovementioned embodiments.

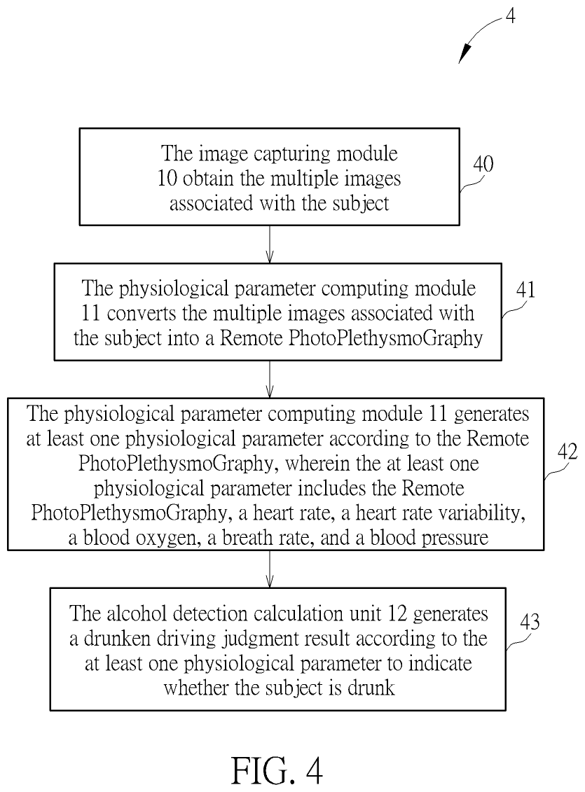

Operations of the drunken driving judgment system 1 may be summarized into a drunken driving judgment process 4, as shown in FIG. 4, the drunken driving judgment process 4 includes the following steps.

Step 40: The image capturing module 10 obtain the multiple images associated with the subject.

Step 41: The physiological parameter computing module 11 converts the multiple images associated with the subject into a Remote PhotoPlethysmoGraphy.

Step 42: The physiological parameter computing module 11 generates at least one physiological parameter according to the Remote PhotoPlethysmoGraphy, wherein the at least one physiological parameter includes the Remote PhotoPlethysmoGraphy, a heart rate, a heart rate variability, a blood oxygen, a breath rate, and a blood pressure.

Step 43: The alcohol detection calculation unit 12 generates a drunken driving judgment result according to the at least one physiological parameter to indicate whether the subject is drunk.

Detailed operations of the drunken driving judgment process 4 may be obtained by referring to descriptions regarding FIG. 1 to FIG. 3, which is omitted.

To sum up, the present invention converts images of the subject into a Remote PhotoPlethysmoGraphy to analyze the physiological parameter including the heart rate, the heart rate variability, the blood oxygen, the breath rate, the blood pressure, and the like, so as to judge whether the subject is drunken. As a result, under the architecture of the drunken driving judgment system, the present invention is able to quickly and easily examine whether the driver is drunken without touching the driver.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A drunken driving judgment system, comprising:
an image capturing module configured to obtain multiple images associated with a subject;
a physiological parameter computing module coupled to the image capturing module, and configured to generate at least one physiological parameter according to the multiple images associated with the subject, wherein the at least one physiological parameter comprises at least one of a Remote PhotoPlethysmoGraphy, a heart rate, a heart rate variability, a blood oxygen, a breath rate, and a blood pressure; and
an alcohol detection calculation unit coupled to the physiological parameter computing module, and configured to generate a drunken driving judgment result according to the at least one physiological parameter to indicate whether the subject is drunken.

2. The drunken driving judgment system of claim 1, wherein the physiological parameter computing module comprises:
a PhotoPlethysmoGraphy conversion module coupled to the image capturing module, and configured to convert the multiple images associated with the subject into the Remote PhotoPlethysmoGraphy;
a heart rate analysis module coupled to the PPG conversion module, and configured to judge the heart rate of the subject according to the Remote PhotoPlethysmoGraphy; and
a heart rate variability analysis module coupled to the PPG conversion module, and configured to judge the heart rate variability of the subject according to the Remote PhotoPlethysmoGraphy.

3. The drunken driving judgment system of claim 1, wherein the heart rate variability comprises at least one time domain indication, the at least one time domain indication comprising a standard deviation of all normal to normal intervals (SDNN) , a root mean square successive differences (RMSSD) and a ratio of P20 to P50, wherein P20/P50 refers to a number of adjacent NN (normal to normal) intervals differing by more than 20/50 milliseconds.

4. The drunken driving judgment system of claim 1, wherein the heart rate variability comprising at least one frequency domain indication, the at least one frequency domain indication comprising a low frequency indication, a high frequency indication and a ratio of the low frequency and the high frequency.

5. The drunken driving judgment system of claim 1, wherein the alcohol detection calculation unit establishes a drunken driving prediction principle using a fuzzy theory according characteristics of the at least one physiological parameter generated by the physiological parameter computing module, and the at least one physiological parameter is inputted to the drunken driving prediction principle to generate the drunken driving judgment result.

6. The drunken driving judgment system of claim 1, wherein the alcohol detection calculation unit establishes a drunken driving prediction model using artificial neural network algorithm according to multiple kinds of learning samples, and the at least one physiological parameter is inputted to the drunken driving prediction model to generate the drunken driving judgment result.

7. A drunken driving judgment method, comprising:
obtaining multiple images associated with a subject;
inputting the multiple images associated with the subject to a physiological parameter computing module to generate at least one physiological parameter, wherein the at least one physiological parameter comprises at least one of a Remote PhotoPlethysmoGraphy, a heart rate, a heart rate variability, a blood oxygen, a breath rate, and a blood pressure; and
inputting the at least one physiological parameter to an alcohol detection calculation unit to generate a drunken driving judgment result to indicate whether the subject is drunken.

8. The drunken driving judgment method of claim 7, wherein inputting the multiple images associated with the subject to the physiological parameter computing module to generate the at least one physiological parameter comprises:
converting the multiple images associated with the subject into the Remote PhotoPlethysmoGraphy;
judging the heart rate of the subject according to the Remote PhotoPlethysmoGraphy; and
judging the heart rate variability of the subject according to the Remote PhotoPlethysmoGraphy.

9. The drunken driving judgment method of claim 7, wherein the heart rate variability comprises at least one time domain indication, the at least one time domain indication comprising a standard deviation of all normal to normal intervals (SDNN) , a root mean square successive differences (RMSSD) and a ratio of P20 to P50, wherein P20/P50 refers to a number of adjacent NN (normal to normal) intervals differing by more than 20/50 milliseconds.

10. The drunken driving judgment method of claim 7, wherein the heart rate variability comprising at least one frequency domain indication, the at least one frequency domain indication comprising a low frequency indication, a high frequency indication and a ratio of the low frequency and the high frequency.

11. The drunken driving judgment method of claim 7, wherein inputting the at least one physiological parameter to the alcohol detection calculation unit to generate the drunken driving judgment result comprises:
establishing a drunken driving prediction principle using a fuzzy theory according characteristics of the at least one physiological parameter generated by the physiological parameter computing module; and
inputting the at least one physiological parameter to the drunken driving prediction principle to generate the drunken driving judgment result.

12. The drunken driving judgment method of claim 7, wherein inputting the at least one physiological parameter to the alcohol detection calculation unit to generate the drunken driving judgment result comprises:
establishing a drunken driving prediction model using artificial neural network algorithm according to multiple kinds of learning samples; and
inputting the at least one physiological parameter to the drunken driving prediction model to generate the drunken driving judgment result.
